(19) United States
(12) Patent Application Publication
(10) Pub. No.: US 2016/0116464 A1
(43) Pub. Date: Apr. 28, 2016

(54) STIMULI-RESPONSIVE MAGNETIC NANOPARTICLES

(71) Applicants: UNIVERSITY OF WASHINGTON THROUGH ITS CENTER FOR COMMERCIALIZATION, Seattle, WA (US); NEXGENIA, INC., Redmond, WA (US)

(72) Inventors: Patrick S. Stayton, Seattle, WA (US); Jriuan Lai, Seattle, WA (US); Dagmara Monfort, Seattle, WA (US); Barrett James Nehilla, Seattle, WA (US)

(73) Assignees: University of Washington through its Center for Commercialization, Seattle, WA (US); Nexgenia, Inc., Redmond, WA (US)

(21) Appl. No.: 14/893,926

(22) PCT Filed: May 29, 2014
(86) PCT No.: PCT/US14/40038

§ 371 Date: Nov. 24, 2015

Related U.S. Application Data
(60) Provisional application No. 61/828,268, filed on May 29, 2013.

(51) Int. Cl. G01N 33/543

(52) U.S. Cl. CPC G01N 33/54333; G01N 2446/20; G01N 2446/84

(57) ABSTRACT

The present disclosure provides stimuli-responsive magnetic nanoparticles, methods of making the magnetic nano-particles, and methods of using the magnetic nanoparticles. The magnetic nanoparticles include a metal oxide core; and a shell that includes a stimuli-responsive polymer having a terminal group that directly coordinates to the metal oxide core. The stimuli-responsive polymer does not include a micelle-forming group, at least at a proximal terminus of the polymer, with respect to the metal oxide core.

### CROSS-REFERENCE TO RELATED APPLICATION

This application claims the benefit of U.S. Patent Application Ser. No. 61/828,268, filed May 29, 2013, the disclosure of which is hereby incorporated by reference in its entirety.

### STATEMENT OF GOVERNMENT LICENSE RIGHTS

This invention was made with government support under EB000252 and under R44 GM100558, both awarded by the National Institutes of Health. The government has certain rights in the invention.

### BACKGROUND

There has been considerable recent interest in the development of magnetic nanoparticle (mNP) technologies for diagnostic and imaging applications. Compared to larger magnetic particles, the smaller nanoparticles (NPs) display potential advantages in their diffusive and superparamagnetic properties. Magnetophoretic mobility, μₘ, is defined as the acceleration of an object in the presence of a magnetic field, which determines the ability to control the object's movement within a magnetic field. The μₘ for an individual particle at room temperature and above is defined as

$${μ}_{m} = \frac{π {μ}_{0} {M}_{S , C}^{2} {D}_{C}^{5}}{324 {k}_{B} T η}$$

where μ₀ is the magnetic constant, M_{S,C} is the saturation magnetic moment of the mNPs, D_{C} is the diameter of the mNPs, k_{B} is the Boltzmann constant, η is the viscosity of the medium, and T is the temperature. Because of their small particle size, which results in randomized magnetic moments, the μₘ for mNPs is usually small. This leads to an intrinsic challenge for applications where the favorable diffusive properties of the small mNPs are advantageous, for example, where the mNPs are used to capture diagnostic targets via antibody-antigen interactions. On the one hand, the small particles display better association and binding properties, but on the other hand their small size reduces magnetic capture efficiency.

Approaches to overcoming the small μₘ for mNPs include using larger macromolecules or objects that are labeled with multiple mNPs, making the mNPs from materials with larger M_{S,C}, using irreversibly aggregated mNPs, or using high magnetic gradients. However, most of these approaches result in the loss of favorable diffusive properties. Furthermore, high magnetic gradients require a large number of coils and high current. Materials with high M_{S,C} are usually metals or alloys and, because of their high surface/volume ratio, they are prone to oxidation events that can lower their M_{S,C}. The pre-aggregation of mNPs into larger structures results in the permanent lowering of surface/volume ratio and to a decrease in the mNPs favorable diffusive properties. There is a need for mNPs with favorable diffusive properties that can also be readily separated in a small magnetic field.

Stimuli-responsive (“intelligent” or “smart”) materials and molecules exhibit abrupt property changes in response to small changes in external stimuli such as pH; temperature; UV-visible light; ionic strength; the concentration of certain chemicals, such as polyvalent ions, polyions of either charge, or enzyme substrates, such as glucose; as well as upon photo-irradiation or exposure to an electric field. Normally these changes are fully reversible once the stimulus has been removed.

Poly(N-isopropylacrylamide) (PNIPAAm) is a temperature-responsive polymer that exhibits a lower critical solution temperature (LCST) around which the polymer reversibly aggregates. Below the LCST, PNIPAAm chains hydrate to form an expanded structure; above the LCST, PNIPAAm chains dehydrate to form a shrinkage structure. This property is due to the thermally-reversible interaction of water molecules with the hydrophobic groups, especially the isopropyl groups, leading to low entropy, hydrophobically-bound water molecules below the LCST and release of those water molecules at and above the LCST. Modification of mNPs with PNIPAAm yields nanoparticles that can be reversibly aggregated in solution as the temperature is cycled through the LCST.

Previous work with PNIPAAm-modified mNPs has relied on post-synthesis chemical modification of the particles. Chiu et al. synthesized a Fe₃O₄ ferrofluid by co-precipitating FeCl₃ and FeCl₂. The ferrofluid was then mixed with a PNIPAAm solution and crosslinked to form magnetic polymeric networks. Lin, C. L. and W. Y. Chiu, J. Polym. Sci., Part A: Polym. Chem. 43, 5923-5934, 2005. Wang et al. also co-precipitated FeCl₃ and FeCl₂ to synthesize Fe₃O₄ particles. Deng, Y., et al., Adv. Mater. 15, 1729-1732, 2003. The particles were coated with a layer of silica and modified with 3-aminopropyltrimethoxysilane to seed the precipitation polymerization of NIPAAm. In both methods, the post-synthesis functionalization requires multiple steps and can result in particle aggregation. There is a need for methods of making stimuli-responsive polymer-modified mNPs that do not require extensive post-synthesis workup steps and result in minimal particle aggregation.

Previous synthetic strategies for stimuli-responsive mNPs via have also included thermal decomposition of an iron precursor, Fe(CO)₅, within a micelle. For example, Fe(CO)₅ was injected into a tetraglyme solution, containing 3.6 mM PNIPAAm formed into micelles, at 100° C. PNIPAAm (ca. 5 kDa) with a terminal micelle-forming moiety was synthesized using reversible fragmentation chain-transfer polymerization with 2-(dodecylsulfanylthiocarbonylsulfanyl)-2-methylpropionic acid (DMP) chain transfer agent (see, e.g., Lai et al., Langmuir. 23(13): 7385-7391, 2007). After iron precursor injection into the tetraglyme solution containing the PNIPAAm micelles, the solution was raised to 190° C. and refluxed for 6 hours before purification to afford stimuli-responsive mNPs that have micelle-forming groups proximally situated to the metal oxide core of the stimuli-responsive mNPs. See, e.g., U.S. Pat. Nos. 8,507,283 and 7,981,688.

Stimuli-responsive materials and molecules have numerous possible applications in the biomedical/pharmaceutical field, as well as in biotechnology and the related industries. Smart conjugates, smart surfaces, smart polymeric micelles, and smart hydrogels have all been studied for a variety of diagnostics, separations, cell culture, drug delivery, and bioprocess applications.

Despite the development of mNP technologies for diagnostic and imaging applications, there exists a need for a stimuli-responsive mNP with favorable diffusive properties as well as with the ability to be reversibly aggregated into larger structures, and simpler methods for making the nanoparticles. The present disclosure seeks to fulfill these needs and provides further related advantages.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, the present disclosure features a process for making a stimuli-responsive magnetic nanoparticle, including (1) providing a mixture including a solvent having a boiling point of greater than 150° C. at atmospheric pressure, a metal complex including a chelating agent coordinated to a metal cation of an element selected from Fe, Ni, Cr, Co, Gd, Dy, and Mn; and a stimuli-responsive polymer; and (2) heating the mixture to provide a stimuli-responsive magnetic nanoparticle, wherein the stimuli-responsive polymer does not include a terminal micelle-forming group.

In another aspect, the present disclosure features a stimuli-responsive magnetic nanoparticle including a metal oxide core and a shell surrounding the metal oxide core, the shell including a stimuli-responsive polymer that includes a terminal carboxylate group. The terminal carboxylate group is directly coordinated to the metal oxide core and the stimuli-responsive polymer does not include a terminal micelle-forming group.

In yet another aspect, the present disclosure features methods of using the stimuli-responsive magnetic nanoparticle.

### DESCRIPTION OF THE DRAWINGS

The foregoing aspects and many of the attendant advantages of this disclosure will become more readily appreciated as the same become better understood by reference to the following detailed description, when taken in conjunction with the accompanying drawings, wherein:

FIG. 1 is a flow chart of an embodiment of a process of making mNPs of the present disclosure.

FIGS. 2A-2C are schematic illustrations of a method of capturing and releasing an embodiment of mNPs of the present disclosure.

FIGS. 3A-3D are graphs showing structural and functional properties of an embodiment of stimuli-responsive mNPs of the present disclosure. The mNPs include a hydrophilic stimuli-responsive polymer that does not include a micelle-forming group at a proximal terminus of the polymer. FIG. 3A is a graph showing the particle size of six different mNP batches, measured by dynamic light scattering. FIG. 3B is a graph showing a lower critical solution temperature (LCST) of 31° C., a measure of the temperature-responsiveness, of the mNPs. FIG. 3C is a graph showing the stimuli-responsive polymer to Fe mass ratio of mNPs, as measured by thermogravimetric analysis. FIG. 3D is a graph showing the separation efficiency of the mNPs at below (22° C.) and above (37° C.) the LCST.

### DETAILED DESCRIPTION

The present disclosure provides stimuli-responsive magnetic nanoparticles (mNPs), methods of making the mNPs, and methods of using the mNPs. The mNPs include a metal oxide core; and a shell that includes a stimuli-responsive polymer having a terminal group that directly coordinates to the metal oxide core. The stimuli-responsive polymer does not include a micelle-forming group at least at a proximal terminus of the polymer, with respect to the metal oxide core.

The stimuli-responsive polymer of the present disclosure (i.e., without a micelle-forming group at a proximal polymer terminus to the metal oxide core of the mNP, when coordinated to the metal oxide core) provides numerous advantages when compared to a stimuli-responsive polymer that includes a micelle-forming group at a polymer terminus proximal to the metal oxide core of the mNP. As will be demonstrated in Example 1, below, when a stimuli-responsive polymer that does not include a micelle-forming group at any polymer terminus was used in the process of the present disclosure, a mNP with unexpectedly improved properties was generated when compared with a mNP that was made using a stimuli-responsive polymer that includes a micelle-forming group at a polymer terminus. Specifically, mNPs made using stimuli-responsive polymers including a micelle-forming group at a polymer terminus have poor yields, are less uniformly sized, cannot be reproducibly synthesized, and can be undesirably insoluble in aqueous environments. Furthermore, mNPs made with stimuli-responsive polymers having a micelle-forming group at a polymer terminus can have undesirably larger sizes and lower LCST values (i.e., close to room temperature), which render the nanoparticles challenging to manipulate at ambient conditions.

### Definitions

At various places in the present specification, substituents of compounds of the disclosure are disclosed in groups or in ranges. It is specifically intended that the disclosure include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the disclosure, which are, for clarity, described in the context of separate embodiments, can also be provided in combination in a single embodiment.

Conversely, various features of the disclosure which are, for brevity, described in the context of a single embodiment, can also be provided separately or in any suitable subcombination.

As used herein, the term “metal complex” refers to a metal-containing compound that includes a central metal atom or ion and a surrounding array of bound molecules or ions (i.e., ligands).

As used herein, the term “coordinate” or “coordinating” refers to the bonds that form between ligands (e.g., chelating agents) and a central metal atom, where the ligands are generally bound to the central atom by donating electrons from a lone electron pair into an empty metal orbital, such that the ligands are coordinated to the atom. In some embodiments, instead of donating electrons from a lone electron pair into an empty metal orbital, pi-bonds of organic ligands such as alkenes can coordinate to empty metal orbitals.

As used herein, the term “chelating agent” refers to a compound that can form two or more separate coordinate bonds to a central atom.

As used herein, the term “hydrodynamic diameter” refers to the apparent size of soluble stimuli-responsive mNPs hydrated in a solvent (e.g., water), as measured by dynamic light scattering.

As used herein, the term “copolymer” refers to a polymer that is the result of polymerization of two or more different monomers. The number and the nature of each constitutional unit can be separately controlled in a copolymer. The constitutional units can be disposed in a purely random, an alternating random, a regular alternating, a regular block, or a random block configuration unless expressly stated to be otherwise. A purely random configuration can, for example, be: x-x-y-z-x-y-y-z-y-z-z-z . . . or y-z-x-y-z-y-z-x-x . . . . An alternating random configuration can be: x-y-x-z-y-x-y-z-y-x-z . . . , and a regular alternating configuration can be: x-y-z-x-y-z-x-y-z . . . . A regular block configuration has the following general configuration: . . . x-x-x-y-y-y-z-z-z-x-x-x . . . , while a random block configuration has the general configuration: . . . x-x-x-z-z-x-x-y-y-y-y-z-z-z-x-x-z-z-z- . . . .

As used herein, the term “substituted” or “substitution” is meant to refer to the replacing of a hydrogen atom with a substituent other than H. For example, an “N-substituted piperidin-4-yl” refers to replacement of the H atom from the NH of the piperidinyl with a non-hydrogen substituent such as, for example, alkyl.

As used herein, the term “alkyl” refers to a straight or branched chain fully saturated (no double or triple bonds) hydrocarbon (carbon and hydrogen only) group. Examples of alkyl groups include, but are not limited to, methyl, ethyl, propyl, isopropyl, butyl, isobutyl, sec-butyl, tertiary butyl, pentyl and hexyl. As used herein, “alkyl” includes “alkylene” groups, which refer to straight or branched fully saturated hydrocarbon groups having two rather than one open valences for bonding to other groups. Examples of alkylene groups include, but are not limited to methylene, —CH₂—, ethylene, —CH₂CH₂—, propylene, —CH₂CH₂CH₂—, n-butylene, —CH₂CH₂CH₂CH₂—, sec-butylene, and —CH₂CH₂CH(CH₃)—. An alkyl group of this disclosure may optionally be substituted with one or more fluorine groups.

As used herein, the term “aryl” refers to monocyclic or polycyclic (e.g., having 2, 3 or 4 fused rings) aromatic hydrocarbons such as, for example, phenyl, naphthyl, anthracenyl, phenanthrenyl, indanyl, and indenyl. In some embodiments, aryl groups have from 6 to about 20 carbon atoms.

As used herein, the term “halo” or “halogen” includes fluoro, chloro, bromo, and iodo.

As used herein, the term “fatty acid” refers to a molecule having a carboxylic acid with a long aliphatic chain, which is either saturated or unsaturated.

As used herein, the term “hydrophobic block” refers to a polymer block that includes more hydrophobic constitutional units than hydrophilic constitutional units. Hydrophobic constitutional units are not ionizable in typical aqueous conditions and include one or more hydrophobic moieties (e.g., alkyl group, aryl group, etc.).

As used herein, the term “constitutional unit” of a polymer refers an atom or group of atoms in a polymer, comprising a part of the chain together with its pendant atoms or groups of atoms, if any. The constitutional unit can refer to a repeat unit. The constitutional unit can also refer to an end group on a polymer chain. For example, the constitutional unit of polyethylene glycol can be —CH₂CH₂O— corresponding to a repeat unit, or —CH₂CH₂OH corresponding to an end group.

As used herein, the term “repeat unit” corresponds to the smallest constitutional unit, the repetition of which constitutes a regular macromolecule (or oligomer molecule or block)

As used herein, the term “end group” refers to a constitutional unit with only one attachment to a polymer chain, located at the end of a polymer. For example, the end group can be derived from a monomer unit at the end of the polymer, once the monomer unit has been polymerized. As another example, the end group can be a part of a chain transfer agent or initiating agent that was used to synthesize the polymer.

As used herein, the term “terminus” of a polymer refers to a constitutional unit of the polymer that is positioned at the end of a polymer backbone.

As used herein, the term “proximal terminus” of a polymer refers to a constitutional unit of the polymer that is positioned at the end of a polymer backbone that coordinates to a metal oxide core of a mNP, once the mNP is formed using the process described herein. The constitutional unit at the end of the polymer backbone (i.e., end group) can be, for example, derived from a monomer unit at the end of the polymer (once the monomer unit has been polymerized), or the end group can be a part of a chain transfer agent or initiating agent that was used to synthesize the polymer.

As used herein, the term “distal terminus” of a polymer refers to a constitutional unit that is positioned at the end of a polymer backbone that is situated away from the proximal terminus of the polymer. In some embodiments, the polymer can have more than one distal termini, such as in the case of a branched polymer, where the distal termini correspond to all the ends of the polymer backbone that are situated away from the proximal terminus of the polymer.

As used herein, the term “micelle-forming group” refers to a group that is capable of forming a micelle in a polar solvent.

As used herein, the term “stimuli-responsive” refers to a material that can respond to changes in external stimuli such as the pH, temperature, UV-visible light, photo-irradiation, exposure to an electric field, ionic strength, and the concentration of certain chemicals by exhibiting property change.

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Process of Making mNPs

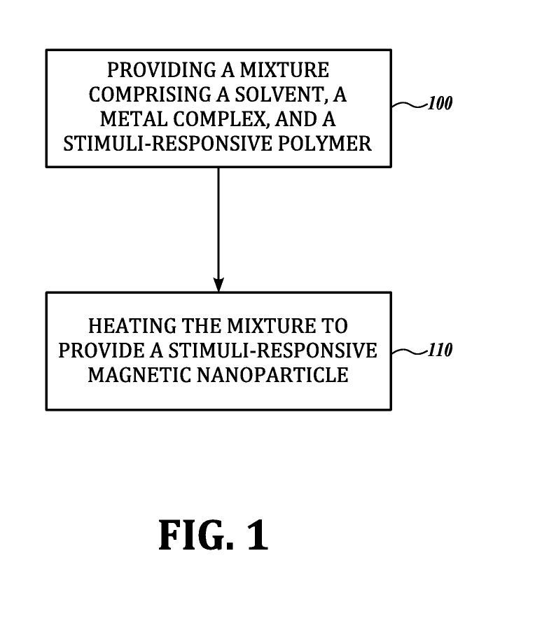

In one aspect, a process for making a stimuli-responsive mNP is provided. Referring to FIG. 1, the process includes a step 100: providing a mixture that includes a solvent, a metal complex, and a stimuli-responsive polymer; and a step 110: heating the mixture to provide a stimuli-responsive mNP. The stimuli-responsive mNP include a metal oxide core; and a shell that includes a stimuli-responsive polymer having a terminal group that directly coordinates to the metal oxide core. The stimuli-responsive polymer does not include a micelle-forming group at least a polymer terminus that is proximal to metal oxide core, when the stimuli-responsive is coordinated to the stimuli-responsive mNP.

The mixture of the first step 100 will now be described in greater detail. The mixture includes a solvent. In one embodiment, the solvent has a boiling point of greater than 150° C. at atmospheric pressure. In some embodiments, the solvent has a polarity index of greater than 2.4. In certain embodiments, the solvent is relatively polar. Non-limiting examples of suitable solvents include diglyme, triglyme, tetraglyme, acetyl acetone, anisole, benzonitrile, cyclohexanone, N,N-dimethylaniline, N,N-dimethylformamide, dimethylsulfoxide, benzyl alcohol, cyclohexanol, diethylene glycol, n-heptanol, n-octanol, xylene, toluene, or any combination thereof. For example, when a solvent mixture is used, the solvent can include from 0% (e.g., from 10%, from 20%, from 30%, from 40%, or from 45%) to 55% (e.g., to 45%, to 40%, to 30%, to 20%, or to 10%) by volume of one solvent (e.g., xylene and/or toluene) relative to the solvent mixture, so long as the total percentage of all the solvent components is 100%. In some embodiments, a suitable solvent can dissolve the metal complex and/or the stimuli-responsive polymer. In some embodiments, the solvent includes oligoethylene glycol ethers, such as tetraglyme.

The mixture also includes a metal complex. The metal complex includes a chelating agent coordinated to a metal cation, which can be a metal cation of iron (Fe), nickel (Ni), chromium (Cr), cobalt (Co), gadolinium (Gd), dysprosium (Dy), or manganese (Mn). The metal complex includes a chelating agent that has a molecular weight of less than 1000 Da (e.g., less than 750 Da, less than 500 Da, or less than 250 Da). In certain embodiments, the chelating agent is a small molecule. The chelating agent is different from the stimuli-responsive polymer in the mixture. In some embodiments, the chelating agent includes a functional group that can coordinate to the metal cation, such as a carboxylic acid (which can coordinate in the form of a carboxylate), a primary amine, and/or a secondary amine. For example, the chelating agent can include a C₈₋₂₈ fatty acid that can be saturated or unsaturated (i.e., include one or more double or triple bonds), a bipyridine, 4-vinyl pyridine, ethylene diamine, ethylenediaminetetraacetic acid, and derivatives thereof. In some embodiments, the saturated or unsaturated C₈₋₂₈ fatty acid, bipyridine, 4-vinyl pyridine, ethylene diamine, and ethylenediaminetetraacetic acid can each be optionally substituted with 1, 2, 3, or 4 substituents selected from C₁₋₄ alkyl, halo, or haloalkyl. Non-limiting examples of the saturated or unsaturated C₈₋₂₈ fatty acids include palmitoleic acid, sapienic acid, oleic acid, elaidic acid, linoleic acid, arachidonic acid, caprylic acid, capric acid, lauric acid, myristic acid, palmitic acid, stearic acid, arachidic acid, behenic acid, lignoceric acid, and cerotic acid. In some embodiments, the chelating agent is oleic acid.

In some embodiments, the metal complex is generated prior to incorporation into the mixture, for example, in a separate reaction. The metal complex can be isolated from the separate reaction before addition into the mixture. In other embodiments, the metal complex is generated in situ in the mixture, for example, by adding a metal cation (e.g., iron (III), such as FeCl₃) and a chelating agent (e.g., an oleate salt, such as sodium oleate) to the mixture.

In some embodiments, the metal cation is a metal cation of Fe, Ni, Cr, Co, Gd, Mn, or combinations thereof. Non-limiting examples of metal cations include Fe(II), Fe(III), Ni(II), Cr(II), Cr(III), Cr(VI), Co(II), Co(III), Gd(III), Mn(II), Mn(III), Mn(IV), Mn(VI), Dy(III), and combinations thereof. In some embodiments, the metal cation is a metal cation of Fe, Ni, or Co. For example, the metal cation can be Fe(III), Ni(II), or Co(III). In one embodiment, the metal cation is Fe (III).

In certain embodiments, the metal complex is iron oleate (e.g., iron(III) tris(oleate).

In some embodiments, the mixture can include the metal complex at a concentration of from 5 mg/mL of the solvent (e.g., from 20 mg/mL, from 40 mg/mL, from 60 mg/mL, or from 80 mg/mL) to 110 mg/mL (e.g., to 80 mg/mL, to 60 mg/mL, to 40 mg/mL, or to 20 mg/mL).

The mixture also includes a stimuli-responsive polymer. The stimuli-responsive polymer does not form a micelle in the mixture during the process of making the stimuli-responsive mNP of the present disclosure. The stimuli-responsive polymer can be any known stimuli-responsive polymer, provided that the polymer does not include a group that is capable of forming a micelle at the proximal terminus of polymer, or at both the proximal and distal polymer termini. Non-limiting examples of stimuli-responsive polymer that can be used in the process of the present disclosure are described in greater detail, below. In some embodiments, the stimuli-responsive polymer has no micelle-forming group on any terminus. In some embodiments, the stimuli-responsive polymer has no micelle-forming group (e.g., no micelle-forming group on any terminus, as pendant groups, or on the polymer backbone). As will be discussed below in Example 1, it was surprisingly found that when a stimuli-responsive polymer that does not include a micelle-forming group at any polymer terminus was used in the process of the present disclosure, a mNP with unexpectedly better properties was generated when compared to a mNP made using a stimuli-responsive polymer that includes a micelle-forming group at a polymer terminus.

In certain embodiments, the stimuli-responsive polymer includes polymers and copolymers of N-isopropylacrylamide that can be optionally substituted with a terminal functional group (e.g., a proximal terminal group, or any one of a proximal or distal terminal groups) such as a carboxylic acid, a primary amine, a secondary amine, a thiol, a hydroxyl, an aldehyde, a ketone, an azide, a hydrazide, or any combination thereof. The mixture can include the stimuli-responsive polymer at a concentration of from 2 mg/mL (e.g., from 15 mg/mL, from 30 mg/mL, from 45 mg/mL, or from 60 mg/mL) to 75 mg/mL (e.g., to 72 mg/mL, to 60 mg/mL, to 45 mg/mL, to 30 mg/mL, or to 15 mg/mL).

Stimuli-Responsive Polymer

The stimuli-responsive polymer can be any polymer having a stimuli-responsive property, provided that the polymer does not include a group that is capable of forming a micelle at the proximal polymer terminus, or at both the proximal and distal polymer termini. The stimuli-responsive polymer can be any one of a variety of polymers that change their associative properties (e.g., change from hydrophilic to hydrophobic) in response to a stimulus. The stimuli-responsive polymer responds to changes in external stimuli such as the pH, temperature, UV-visible light, photo-irradiation, exposure to an electric field, ionic strength, and the concentration of certain chemicals by exhibiting property change. The chemicals could be polyvalent ions such as calcium ion, polyions of either charge, or enzyme substrates such as glucose. For example, the temperature-responsive polymer is responsive to changes in temperature by exhibiting a LCST in aqueous solution. The stimuli-responsive polymer can be a multi-responsive polymer, where the polymer exhibits property change in response to combined simultaneous or sequential changes in two or more external stimuli.

The stimuli-responsive polymers may be synthetic or natural polymers that exhibit reversible conformational or physico-chemical changes such as folding/unfolding transitions, reversible precipitation behavior, or other conformational changes to in response to stimuli, such as to changes in temperature, light, pH, ions, or pressure. Representative stimuli-responsive polymers include temperature-sensitive polymers, pH-sensitive polymers, and light-sensitive polymers.

Stimulus-responsive polymers useful in making the nanoparticle described herein can be any which are sensitive to a stimulus that causes significant conformational changes in the polymer. Illustrative polymers described herein include temperature-, pH-, ion- and/or light-sensitive polymers. Hoffman, A. S., “Intelligent Polymers in Medicine and Biotechnology”, Artif. Organs. 19:458-467, 1995; Chen, G. H. and A. S. Hoffman, “A New Temperature- and Ph-Responsive Copolymer for Possible Use in Protein Conjugation”, Macromol. Chem. Phys. 196:1251-1259. 1995; Irie, M. and D. Kungwatchakun, “Photoresponsive Polymers. Mechanochemistry of Polyacrylamide Gels Having Triphenylmethane Leuco Derivatives”, Makromol. Chem., Rapid Commun. 5:829-832, 1985; and Irie, M., “Light-induced Reversible Conformational Changes of Polymers in Solution and Gel Phase”, ACS Polym. Preprints, 27(2):342-343, 1986; each of which is incorporated by reference herein.

Stimuli-responsive oligomers and polymers useful in the nanoparticle described herein can be synthesized that range in molecular weight from about 1,000 to 50,000 Daltons. In one embodiment, these syntheses are based on the chain transfer-initiated free radical polymerization of vinyl-type monomers, as described herein, and by (1) Tanaka, T., “Gels”, Sci. Amer. 244:124-138. 1981; (2) Osada, Y. and S. B. Ross-Murphy, “Intelligent Gels”, Sci. Amer, 268:82-87, 1993; (3) Hoffman, A. S., “Intelligent Polymers in Medicine and Biotechnology”, Artif. Organs 19:458-467, 1995; also Macromol. Symp. 98:645-664, 1995; (4) Feijen, J., et al., “Thermosensitive Polymers and Hydrogels Based on N-isopropylacrylamide”, 11th European Conf. on Biomtls:256-260, 1994; (5) Monji, N. and A. S. Hoffman, “A Novel Immunoassay System and Bioseparation Process Based on Thermal Phase Separating Polymers”, Appl. Biochem. and Biotech. 14:107-120, 1987; (6) Fujimura, M., T. Mori and T. Tosa, “Preparation and Properties of Soluble-Insoluble Immobilized Proteases”, Biotech. Bioeng. 29:747-752, 1987; (7) Nguyen, A. L. and J. H. T. Luong, “Synthesis and Applications of Water-Soluble Reactive Polymers for Purification and Immobilization of Biomolecules”, Biotech. Bioeng. 34:1186-1190, 1989; (8) Taniguchi, M., et al., “Properties of a Reversible Soluble-Insoluble Cellulase and Its Application to Repeated Hydrolysis of Crystalline Cellulose”, Biotech. Bioeng. 34:1092-1097, 1989; (9) Monji, N., et al., “Application of a Thermally-Reversible Polymer-Antibody Conjugate in a Novel Membrane-Based Immunoassay”, Biochem. and Biophys. Res. Comm. 172:652-660, 1990; (10) Monji, N. C. A. Cole, and A. S. Hoffman, “Activated, N-Substituted Acrylamide Polymers for Antibody Coupling: Application to a Novel Membrane-Based Immunoassay”, J. Biomtls. Sci. Polymer Ed. 5:407-420, 1994; (11) Chen, J. P. and A. S. Hoffman, “Polymer-Protein Conjugates: Affinity Precipitation of Human IgG by Poly(N-Isopropyl Acrylamide)-Protein A Conjugates”, Biomtls. 11:631-634, 1990; (12) Park, T. G. and A. S. Hoffman, “Synthesis and Characterization of a Soluble, Temperature-Sensitive Polymer-Conjugated Enzyme, J. Biomtls. Sci. Polymer Ed. 4:493-504, 1993; (13) Chen, G. H., and A. S. Hoffman, Preparation and Properties of Thermo-Reversible, Phase-Separating Enzyme-Oligo(NIPAAm) Conjugates”, Bioconj. Chem. 4:509-514, 1993; (14) Ding, Z. L., et al., “Synthesis and Purification of Thermally-Sensitive Oligomer-Enzyme Conjugates of Poly(NIPAAm)-Trypsin”, Bioconj. Chem. 7: 121-125, 1995; (15) Chen, G. H. and A. S. Hoffman, “A New Temperature- and pH-Responsive Copolymer for Possible Use in Protein Conjugation”, Macromol. Chem. Phys. 196:1251-1259, 1995; (16) Takei, Y. G., et al., “Temperature-responsive Bioconjugates. 1. Synthesis of Temperature-Responsive Oligomers with Reactive End Groups and their Coupling to Biomolecules”, Bioconj. Chem. 4:42-46, 1993; (17) Takei, Y. G., et al., “Temperature-responsive Bioconjugates. 2. Molecular Design for Temperature-modulated Bioseparations”, Bioconj. Chem. 4:341-346, 1993; (18) Takei, Y. G., et al., “Temperature-responsive Bioconjugates. 3. Antibody-Poly(N-Isopropylamide) Conjugates for Temperature-Modulated Precipitations and Affinity Bioseparations”, Bioconj. Chem. 5:577-582, 1994; (19) Matsukata, M., et al., “Temperature Modulated Solubility-Activity Alterations for Poly(N-Isopropylacrylamide)-Lipase Conjugates”, J. Biochem. 116:682-686, 1994; (20) Chilkoti, A., et al., “Site-Specific Conjugation of a Temperature-Sensitive Polymer to a Genetically-Engineered Protein”, Bioconj. Chem. 5:504-507, 1994; and (21) Stayton, P. S., et al., “Control of Protein-Ligand Recognition Using a Stimuli-Responsive Polymer”, Nature 378:472-474, 1995.

The stimuli-responsive polymers useful in the nanoparticles of the disclosure include homopolymers and copolymers having stimuli-responsive behavior. Other suitable stimuli-responsive polymers include block and graft copolymers having one or more stimuli-responsive polymer components. A suitable stimuli-responsive block copolymer may include, for example, a temperature-sensitive polymer block, or a pH-sensitive block. A suitable stimuli-responsive graft copolymer may include, for example, a pH-sensitive polymer backbone and pendant temperature-sensitive polymer components, or a temperature-sensitive polymer backbone and pendant pH-sensitive polymer components. Polymers having temperature-sensitive or pH-sensitive components can be hydrophilic below their LCST's and do not have micelle-forming groups at the proximal terminus, or at both the proximal and distal termini.

The stimuli-responsive polymer can include a polymer having a balance of hydrophilic and hydrophobic groups, such as polymers and copolymers of N-isopropylacrylamide. An appropriate hydrophilic/hydrophobic balance in a smart vinyl type polymer is achieved, for example, with a pendant hydrophobic group of about 2-6 carbons that hydrophobically bond with water, and a pendant polar group such as an amide, acid, amine, or hydroxyl group that H-bond with water. Other polar groups include sulfonate, sulfate, phosphate and ammonium ionic groups. Preferred embodiments are for 3-4 carbons (e.g., propyl, isopropyl, n-butyl, isobutyl, and t-butyl) combined with an amide group (e.g., PNIPAAm), or 2-4 carbons (e.g., ethyl, propyl, isopropyl, n-butyl, isobutyl, and t-butyl) combined with a carboxylic acid group (e.g., PPAA). There is also a family of smart A-B-A (also A-B-C) block copolymers of polyethers, such as PLURONIC polymers having compositions of PEO-PPO-PEO, or polyester-ether compositions such as PLGA-PEG-PLGA. In one embodiment, the stimuli-responsive polymer is a temperature responsive polymer, poly(N-isopropylacrylamide) (PNIPAAm). Polymers having a balance of hydrophilic and hydrophobic groups can be hydrophilic below their LCST's and do not have micelle-forming groups at the proximal terminus, or at both the proximal and distal termini.

The stimuli-responsive polymer useful in the disclosure can be a smart polymer having different or multiple stimuli-responsivities, such as homopolymers responsive to pH or light. Block, graft, or random copolymers with dual sensitivities, such as pH and temperature, light and temperature, or pH and light, may also be used.

Temperature-Sensitive Polymers

Illustrative embodiments of the many different types of temperature-sensitive polymers that may be conjugated to interactive molecules are polymers and copolymers of N-isopropylacrylamide (NIPAAm). PNIPAAm is a thermally sensitive polymer that precipitates out of water at 32° C., which is its lower critical solution temperature (LCST), or cloud point (Heskins and Guillet, J. Macromol. Sci.-Chem. A2:1441-1455, 1968). When PNIPAAm is copolymerized with more hydrophilic comonomers such as acrylamide, the LCST is raised. The opposite occurs when it is copolymerized with more hydrophobic comonomers, such as N-t-butyl acrylamide. Copolymers of NIPAAm with more hydrophilic monomers, such as AAm, have a higher LCST, and a broader temperature range of precipitation, while copolymers with more hydrophobic monomers, such as N-t-butyl acrylamide, have a lower LCST and usually are more likely to retain the sharp transition characteristic of PNIPAAm (Taylor and Cerankowski, J. Polymer Sci. 13:2551-2570, 1975; Priest et al., ACS Symposium Series 350:255-264, 1987; and Heskins and Guillet, J. Macromol. Sci.-Chem. A2:1441-1455, 1968, the disclosures of which are incorporated herein). Copolymers can be produced having higher or lower LCSTs and a broader temperature range of precipitation. Polymers and copolymers of N-isopropylacrylamide of the present disclosure can have varying proportions of hydrophilic and hydrophobic comonomers, but do not have micelle-forming groups at the proximal terminus, or at both the proximal and distal termini.

The synthesis of an amino-terminated polymer proceeds by the radical polymerization of NIPAAm in the presence of AIBN as an initiator and 1-aminoethanethiol-hydrochloride as a chain transfer reagent. To synthesize a chain with —COOH or —OH terminal groups, carboxyl- or hydroxyl-thiol chain transfer agents, respectively, have been used instead of the amino-thiol. It should be noted that the synthesis of the end-reactive polymers is based on a chain transfer initiation and termination mechanism. This yields a relatively short polymer chain, having a molecular weight somewhere between 1000 Da and 25,000 Da to 30,000 Da. The shortest chains, less than 10,000 in molecular weight, are usually called “oligomers.” Oligomers of different molecular weights can be synthesized by simply changing the ratio of monomer to chain transfer reagent, and controlling their concentration levels, along with that of the initiator. The polymers useful in the disclosure may also be prepared by reversible addition fragmentation chain transfer (RAFT) polymerization.

Oligomers of NIPAAm (or other vinyl monomers) having a reactive group at one end are prepared by the radical polymerization of NIPAAm using AIBN as initiator, plus a chain transfer agent with a thiol (—SH) group at one end and the desired “reactive” group (e.g., —OH, —COOH, —NH₂) at the other end. Chen and Hoffman, Bioconjugate Chem. 4:509-514, 1993 and Chen and Hoffman, J. Biomaterials Sci. Polymer Ed. 5:371-382, 1994, each of which is incorporated herein by reference. The oligomers of NIPAAm do not have a micelle-forming group at the proximal terminus, or at both the proximal and distal termini. Appropriate quantities of NIPAAm, AIBN and chain transfer reagent in DMF are placed in a thick-walled polymerization tube and the mixtures are degassed by freezing and evacuating and then thawing (4 times). After cooling for the last time, the tubes are evacuated and sealed prior to polymerization. The tubes are immersed in a water bath at 60° C. for 4 h. The resulting polymer is isolated by precipitation into diethyl ether and weighed to determine yield. The molecular weight of the polymer is determined either by titration (if the end group is amine or carboxyl), by vapor phase osmometry (VPO), or gel permeation chromatography (GPC).

In some embodiments, temperature sensitive oligopeptides also may be incorporated into the nanoparticles.

pH-Sensitive Polymers

Synthetic pH-sensitive polymers useful in making the nanoparticles described herein are typically based on pH-sensitive vinyl monomers, such as acrylic acid (AAc), methacrylic acid (MAAc) and other alkyl-substituted acrylic acids such as ethylacrylic acid (EAAc), propylacrylic acid (PAAc), and butylacrylic acid (BAAc), maleic anhydride (MAnh), maleic acid (MAc), AMPS (2-acrylamido-2-methyl-1-propanesulfonic acid), N-vinyl formamide (NVA), N-vinyl acetamide (NVA) (the last two may be hydrolyzed to polyvinylamine after polymerization), aminoethyl methacrylate (AEMA), phosphoryl ethyl acrylate (PEA) or methacrylate (PEMA). pH-Sensitive polymers may also be synthesized as polypeptides from amino acids (e.g., polylysine or polyglutamic acid) or derived from naturally-occurring polymers such as proteins (e.g., lysozyme, albumin, casein), or polysaccharides (e.g., alginic acid, hyaluronic acid, carrageenan, chitosan, carboxymethyl cellulose) or nucleic acids, such as DNA. pH-Responsive polymers usually contain pendant pH-sensitive groups such as —OPO(OH)₂, —COOH, or —NH₂ groups. With pH-responsive polymers, small changes in pH can stimulate phase-separation, similar to the effect of temperature on solutions of PNIPAAm (Fujimura et al., Biotech. Bioeng. 29:747-752, 1987). By randomly copolymerizing a thermally-sensitive NIPAAm with a small amount (e.g., less than 10 mole percent) of a pH-sensitive comonomer such as AAc, a copolymer will display both temperature and pH sensitivity. Its LCST will be almost unaffected, sometimes even lowered a few degrees, at pHs where the comonomer is not ionized, but it will be dramatically raised if the pH-sensitive groups are ionized. When the pH-sensitive monomer is present in a higher content, the LCST response of the temperature sensitive component may be “eliminated” (e.g., no phase separation seen up to and above 100° C.). The pH-sensitive polymers of the present disclosure do not have micelle-forming groups at the proximal terminus, or at both the proximal and distal termini.

Graft and block copolymers of pH and temperature sensitive monomers can be synthesized that retain both pH and temperature transitions independently. Chen, G. H., and A. S. Hoffman, Nature 373:49-52, 1995. For example, a block copolymer having a pH-sensitive block (polyacrylic acid) and a temperature sensitive block (PNIPAAm) can be useful in the disclosure. The graft and block copolymers having both pH and temperature sensitivity of the present disclosure do not have micelle-forming groups at the proximal terminus, or at both the proximal and distal termini.

Light-Sensitive Polymers

Light-responsive polymers usually contain chromophoric groups pendant to or along the main chain of the polymer and, when exposed to an appropriate wavelength of light, can be isomerized from the trans to the cis form, which is dipolar and more hydrophilic and can cause reversible polymer conformational changes. Other light sensitive compounds can also be converted by light stimulation from a relatively non-polar hydrophobic, non-ionized state to a hydrophilic, ionic state.

In the case of pendant light-sensitive group polymers, the light-sensitive dye, such as aromatic azo compounds or stilbene derivatives, may be conjugated to a reactive monomer (an exception is a dye such as chlorophyllin, which already has a vinyl group) and then homopolymerized or copolymerized with other conventional monomers, or copolymerized with temperature-sensitive or pH-sensitive monomers using the chain transfer polymerization as described above. The light sensitive group may also be conjugated to one end of a different (e.g., temperature) responsive polymer. A number of protocols for such dye-conjugated monomer syntheses are known.

Although both pendant and main chain light sensitive polymers may be synthesized and are useful for the methods and applications described herein, the preferred light-sensitive polymers and copolymers thereof are typically synthesized from vinyl monomers that contain light-sensitive pendant groups. Copolymers of these types of monomers are prepared with “normal” water-soluble comonomers such as acrylamide, and also with temperature- or pH-sensitive comonomers such as NIPAAm or AAc. The light-sensitive polymers and copolymer of the present disclosure do not have micelle-forming groups at the proximal terminus, or at both the proximal and distal termini.

Light-sensitive compounds may be dye molecules that isomerize or become ionized when they absorb certain wavelengths of light, converting them from hydrophobic to hydrophilic conformations, or they may be other dye molecules which give off heat when they absorb certain wavelengths of light. In the former case, the isomerization alone can cause chain expansion or collapse, while in the latter case the polymer will precipitate only if it is also temperature-sensitive.

Light-responsive polymers usually contain chromophoric groups pendant to the main chain of the polymer. Typical chromophoric groups that have been used are the aromatic diazo dyes (Ciardelli, Biopolymers 23:1423-1437, 1984; Kungwatchakun and Irie, Macromol. Chem., Rapid Commun. 9:243-246, 1988; Lohmann and Petrak, CRC Crit. Rev. Therap. Drug Carrier Systems 5:263, 1989; Mamada et al., Macromolecules 23:1517, 1990, each of which is incorporated herein by reference). When this type of dye is exposed to 350-410 nm UV light, the trans form of the aromatic diazo dye, which is more hydrophobic, is isomerized to the cis form, which is dipolar and more hydrophilic, and this can cause polymer conformational changes, causing a turbid polymer solution to clear, depending on the degree of dye-conjugation to the backbone and the water solubility of the main unit of the backbone. Exposure to about 750 nm visible light will reverse the phenomenon. Such light-sensitive dyes may also be incorporated along the main chain of the backbone, such that the conformational changes due to light-induced isomerization of the dye will cause polymer chain conformational changes. Conversion of the pendant dye to a hydrophilic or hydrophobic state can also cause individual chains to expand or contract their conformations. When the polymer main chain contains light sensitive groups (e.g., azo benzene dye) the light-stimulated state may actually contract and become more hydrophilic upon light-induced isomerization. The light-sensitive polymers can include polymers having pendant or backbone azobenzene groups.

Specific Ion-Sensitive Polymers

Polysaccharides, such as carrageenan, that change their conformation, for example, from a random to an ordered conformation, as a function of exposure to specific ions, such as potassium or calcium, can also be used as the stimulus-responsive polymers. In another example, a solution of sodium alginate may be gelled by exposure to calcium. Other specific ion-sensitive polymers include polymers with pendant ion chelating groups, such as histidine or EDTA.

Dual- or Multi-Sensitivity Polymers

If a light-sensitive polymer is also thermally-sensitive, the UV- or visible light-stimulated conversion of a chromophore conjugated along the backbone to a more hydrophobic or hydrophilic conformation can also stimulate the dissolution or precipitation of the copolymer, depending on the polymer composition and the temperature. If the dye absorbs the light and converts it to thermal energies rather than stimulating isomerization, then the localized heating can also stimulate a phase change in a temperature-sensitive polymer such as PNIPAAm, when the system temperature is near the phase separation temperature. The ability to incorporate multiple sensitivities, such as temperature and light sensitivity, or temperature and pH sensitivity, along one backbone by vinyl monomer copolymerization lends great versatility to the synthesis and properties of the responsive polymer-protein conjugates. For example, dyes can be used which bind to protein recognition sites, and light-induced isomerization can cause loosening or detachment of the dye from the binding pocket (Bieth et al., Proc. Natl. Acad. Sci. USA 64:1103-1106, 1969). This can be used for manipulating affinity processes by conjugating the dye to the free end of a temperature responsive polymer, such as ethylene oxide-propylene oxide (EO-PO) random copolymers available from Carbide. These polymers, —(CH₂CH₂O)ₓ—(CH₂CHCH₃O)_{y}—, have two reactive end groups. The phase separation point (cloud point) can be varied over a wide range, depending on the EO/PO ratio, molecular weight, and concentration, and one end may be derivatized with the ligand dye and the other end with an —SH reactive group, such as vinyl sulfone (VS).

Referring again to FIG. 1, the process of making the stimuli-responsive nanoparticle further includes a step 110, which includes heating the mixture to provide a stimuli-responsive mNP. The stimuli-responsive mNP produced by the method are of the type described in greater detail below.

In some embodiments, the heating step is performed under an atmosphere that includes oxygen (e.g., ambient atmosphere). In other embodiments, the heating step is performed under an atmosphere that is substantially oxygen-free (e.g., 95% oxygen-free, 97% oxygen-free, 99% oxygen-free or more). The heating step can be to a temperature of between 100° C. and 240° C. (e.g., between 140° C. and 240° C., between 160° C. and 200° C., between 160° C. and 190° C., between 170° C. and 240° C., or between 190° C. and 240° C.). In some embodiments, the heating step includes refluxing the mixture at a temperature corresponding to the boiling point of the solvent. In some embodiments, the heating step can be performed under pressure. For example, the heating step can be performed at a pressure of from atmospheric pressure of 1 bar (e.g., from 50 bar, from 100 bar, from 150 bar) to 200 bar (e.g., to 150 bar, to 100 bar, to 50 bar). The heating step can be performed for a duration of from 1 hour (e.g., from 2 hours, from 4 hours, from 6 hours, or from 8 hours) to 10 hours (e.g., to 8 hours, to 6 hours, to 4 hours, or to 2 hours).

In some embodiments, after the heating step, the stimuli-responsive mNP is isolated by precipitation in a solvent in which the stimuli-responsive mNP is insoluble (e.g., a hydrophobic solvent, such as pentane). The stimuli-responsive mNP can be purified, for example, by tangential flow filtration, by dialysis, or by ultrafiltration.

### Magnetic Nanoparticles

In another aspect, the process above provides a stimuli-responsive mNP that includes a metal oxide core; and a shell surrounding the metal oxide core. The shell includes a stimuli-responsive polymer having a terminal group that directly coordinates to the metal oxide core, such as a carboxylate, a primary amine, and/or a secondary amine. The stimuli-responsive polymer does not include a micelle-forming group at least at a proximal terminus of the polymer.

In some embodiments, the stimuli-responsive nanoparticle includes a core including a magnetic metal oxide formed from the metal cation. Non-limiting examples of magnetic metal oxide include, for example, iron oxide (e.g., ferric oxide, ferrous oxide), nickel oxide, nickel oxide, chromium oxide, gadolinium oxide, dysprosium oxide, and manganese oxide, or any combination thereof.

In some embodiments, the stimuli-responsive polymer is coordinated to the core via a terminal functional group (e.g., carboxylate, primary amine, secondary amine, hydroxyl, an aldehyde, a ketone, an azide, and/or a hydrazide) on the stimuli-responsive polymer. The stimuli-responsive polymer does not include a group (e.g., an alkyl group, aryl group, a hydrophobic copolymer block, a polypeptide, etc.) that is capable of forming a micelle at the proximal polymer terminus to the metal oxide core, or at both the proximal and distal polymer termini to the metal oxide core. In some embodiments, the stimuli-responsive polymer has no micelle-forming group on any terminus. In some embodiments, the stimuli-responsive polymer has no micelle-forming group (e.g., no micelle-forming group on any terminus, as pendant groups, or on the polymer backbone).

In certain embodiments, the stimuli-responsive polymer includes polymers and copolymers of N-isopropylacrylamide, and the polymer and copolymers of N-isopropylacrylamide includes a terminus distal to the metal oxide core having a formula

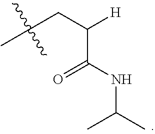 [SMILES: *CCC(=O)NC(C)C]

In some embodiments, the stimuli-responsive mNP includes a stimuli-responsive polymer to metal oxide mass ratio of from 1:1 to 3:1 (e.g., from 2:1 to 3:1, or from 1:1 to 2:1). For example, the polymer to metal oxide mass ratio can be 2:1. The mass ratio of polymer can be determined, for example, by thermogravimetric analysis, from the ratio of stimuli-responsive polymer decomposition mass loss and a remaining mass after polymer removal.

In some embodiments, the stimuli-responsive nanoparticle has a hydrodynamic diameter of from 10 nm (e.g., from 20 nm, from 30 nm, or from 40 nm) to 60 nm (e.g., to 40 nm, to 30 nm, or to 20 nm). For example, the stimuli-responsive nanoparticle can have a hydrodynamic diameter of from 10 nm to 35 nm (e.g., from 15 nm to 30 nm). The stimuli-responsive nanoparticle can respond to a stimulus such as temperature, pH, light, electric field, and/or ionic strength.

### Polymer Capture

Binding Pairs. In some embodiments, the stimuli-responsive mNPs include polymers having distal terminal functional groups for covalently coupling a capture molecule. The terminal functional group on the stimuli-responsive polymer refers to any reactable group that may be derivatized to make it reactive with the capture moiety, such as carboxyl, hydroxyl, and amine groups. The terminal functional group may be derivatized to form reactive groups such as thiol, ketone, N-hydroxy succinimide esters, N-hydroxy maleimide esters, tetrafluorophenyl esters, pentafluorophenyl esters, carbonyl imidazoles, carbodiimide esters, vinyl sulfone, acrylate, benzyl halide, tosylate, tresylate, aldehyde, hydrazide, acid halide, p-nitrophenolic esters, and hydroperoxides. In one embodiment, the terminal functional group on the stimuli-responsive polymer is a carboxylic group.

The terminal functional group on the stimuli-responsive polymer can be coupled with a capture molecule through covalent bonds, including but not limited to amide, esters, ether, thioether, disulfide, hydrazone, acetal, ketal, ketone, anhydride, urethane, urea, and carbamate bonds. In one embodiment, the biotin moiety is coupled to the stimuli-responsive polymer through an amide bond.

The terminal functional group can be covalently coupled to a capture molecule, such as a protein, a nucleic acid oligomer (DNA or RNA), an antibody, an antigen, an enzyme or an enzyme substrate. The capture moiety can be further coupled with a target molecule, such as a protein, a nucleic acid oligomer (DNA or RNA), an antigen, an antibody, an enzyme, or an enzyme substrate through covalent or non-covalent interaction. In one embodiment, the terminal functional group is coupled to a biotin, the capture molecule, to afford a biotinylated nanoparticle. In one embodiment, the biotinylated nanoparticle can be further conjugated to a streptavidin, the target molecule, to yield a streptavidin-conjugated biotinylated nanoparticle that can be coupled to a biotinylated target molecule.

A capture molecule and a target molecule form a binding pair. Each has an affinity toward the other (e.g., antigen and antibody). Each of the capture molecule and the target molecule can be a variety of different molecules, including peptides, proteins, poly- or oligosaccharides, glycoproteins, lipids and lipoproteins, and nucleic acids, as well as synthetic organic or inorganic molecules having a defined bioactivity, such as an antibiotic or anti-inflammatory agent, that binds to a target site, such as a cell membrane receptor. The exemplary proteins include antibodies (monoclonal, polyclonal, chimeric, single-chain or other recombinant forms), their protein/peptide antigens, protein/peptide hormones, streptavidin, avidin, protein A, protein G, growth factors and their respective receptors, DNA-binding proteins, cell membrane receptors, endosomal membrane receptors, nuclear membrane receptors, neuron receptors, visual receptors, and muscle cell receptors. Exemplary oligonucleotides include DNA (genomic or cDNA), RNA, antisense, ribozymes, and external guide sequences for RNAase P, and can range in size from short oligonucleotide primers up to entire genes. Carbohydrates include tumor associated carbohydrates (e.g., Le^{x}, sialyl Le^{x}, Le^{y}, and others identified as tumor associated as described in U.S. Pat. No. 4,971,905, incorporated herein by reference), carbohydrates associated with cell adhesion receptors (e.g., Phillips et al., Science 250:1130-1132, 1990), and other specific carbohydrate binding molecules and mimetics thereof which are specific for cell membrane receptors.

Among the proteins, streptavidin is particularly useful as a model for other capture moiety-target molecule binding pair systems described herein. Streptavidin is an important component in many separations and diagnostic technologies which use the very strong association of the streptavidin-biotin affinity complex. (Wilchek and Bayer, Avidin-Biotin Technology, New York, Academic Press, Inc., 1990; and Green, Meth. Enzymol. 184:51-67. Protein G, a protein that binds IgG antibodies (Achari et al., Biochemistry 31:10449-10457, 1992, and Akerstrom and Bjorck, J. Biol. Chem. 261:10240-10247, 1986) is also useful as a model system. Representative immunoaffinity molecules include engineered single chain Fv antibody (Bird et al., Science 242:423-426, 1988 and U.S. Pat. No. 4,946,778 to Ladner et al., incorporated herein by reference, Fab, Fab′, and monoclonal or polyclonal antibodies.

In one embodiment, the capture molecule is an antibody and the target molecule is an antigen. In another embodiment, both the capture molecule and the target molecule are protein. In another embodiment, the capture molecule is a nucleic acid (DNA or RNA) and the target molecule is a complimentary nucleic acid (DNA or RNA). In another embodiment, the target molecule is a nucleic acid (DNA or RNA) and the capture molecule is a protein. In another embodiment, the capture molecule is a cell membrane receptors and the target molecule is a ligand. In another embodiment, the capture moiety is an enzyme and the target molecule is a substrate. In another embodiment, the capture molecule is biotin and the target molecule is streptavidin or avidin. In another embodiment, the target moiety is a cell (e.g., a living cell).

### Methods for Using the Stimuli-Responsive Nanoparticles

In other aspects, the disclosure provides methods for using the nanoparticle.

In one embodiment, the disclosure provides a method for capturing a target, including:

(a) contacting a medium comprising a target with a plurality of stimuli-responsive mNPs, wherein each nanoparticle comprises a capture moiety reactive toward the target;

(b) applying an external stimulus to provide aggregated nanoparticles;

(c) subjecting the aggregated nanoparticle to a magnetic field to provide magnetically aggregated nanoparticles; and

(d) removing the stimulus and the magnetic field to regenerate the nanoparticles, wherein the regenerated nanoparticles further include the target.

In the above methods, the external stimulus could be temperature, pH, or light. In one embodiment, the stimuli-responsive mNP is a temperature-responsive nanoparticle, and the external stimulus is temperature (e.g., a change in temperature). In one embodiment the stimuli-responsive mNP is a pH-responsive nanoparticle, and the external stimulus is the pH. In one embodiment the stimuli-responsive mNP is a light-responsive nanoparticle, and the external stimulus is light. In one embodiment the stimuli-responsive mNP is ion-responsive nanoparticle, and the external stimulus is the ion strength of a specific ion.

The disclosure provides a method for capturing a diagnostic target, including:

(a) contacting a medium including a diagnostic target with a plurality of temperature-responsive mNPs, wherein each nanoparticle includes a capture moiety reactive toward the diagnostic target;

(b) increasing the temperature of the medium to above the lower critical solution temperature of the nanoparticle to provide thermally aggregated nanoparticles;

(c) subjecting the thermally aggregated nanoparticles to a magnetic field to provide magnetically aggregated nanoparticles; and

(d) decreasing the temperature to below the lower critical solution temperature of the nanoparticle and removing the magnetic field to regenerate the nanoparticles, wherein the regenerated nanoparticles further comprise the diagnostic target.

In one embodiment, the disclosure provides a method for concentrating a diagnostic target, including:

(a) contacting a medium comprising a diagnostic target with a plurality of temperature-responsive mNPs, wherein each nanoparticle includes a capture moiety reactive toward the diagnostic target;

(b) increasing temperature of the medium to above the lower critical solution temperature of the nanoparticle to provide thermally aggregated nanoparticles;

(c) subjecting the thermally aggregated nanoparticles to a magnetic field to provide magnetically aggregated nanoparticles; and

(d) decreasing the temperature to below the lower critical solution temperature of the nanoparticle and removing the magnetic field to regenerate the nanoparticles, wherein the regenerated nanoparticles further comprise the diagnostic target.

In the above methods, steps (b) to (d) may be repeated.

In the above methods, the diagnostic target molecule and the capture moiety each has affinity toward the other and are capable of forming a binding pair. As used herein, the term “diagnostic target” refers to a molecule that is indicative of a diseased condition or an indicator of exposure to a toxin, or a therapeutic drug that has been administered to a subject and whose concentration is to be monitored.

In one embodiment, the diagnostic target molecule is an antibody and the capture moiety is an antigen. In one embodiment, the diagnostic target molecule is an antigen and the capture moiety is an antibody. In one embodiment, the diagnostic target molecule is a nucleic acid oligomer (RNA or DNA) and the capture moiety is a complementary nucleic acid oligomer. In one embodiment, the diagnostic target molecule is a nucleic acid oligomer (RNA or DNA) and the capture moiety is a protein. In one embodiment, the diagnostic target molecule is a protein and the capture moiety is a nucleic acid oligomer (RNA or DNA). In one embodiment, the diagnostic target molecule is an enzyme and the capture moiety is a substrate. In one embodiment, the diagnostic target molecule is an enzyme substrate and the capture moiety is an enzyme.

The dual magneto- and thermally-responsive mNPs of the disclosure are designed to facilitate diagnostic target isolation and/or assay. The temperature responsive mNPs reversibly aggregate as the temperature is cycled above and below the LCST. Aggregation of the mNPs results in an increase of the effective particle size, facilitating the magnetic separation of the nanoparticles with a small applied magnetic field. As the temperature is reversed below the LCST and the applied field is removed, the captured nanoparticles can be recovered quickly. The mNPs can have a separation efficiency that is a measure of how many mNPs could be separated from a solution after exposure to a magnetic field. In some embodiments, absorbance of a supernatant solution at a given wavelength can be measured, a loss of absorbance can be compared to an absorbance of a control mNP solution, and the loss of absorbance can be quantified as the separation efficiency. In some embodiments, the separation efficiency can be 80% or more (e.g., 90% or more, 95% or more, 97% or more, 98% or more, or 99% or more).

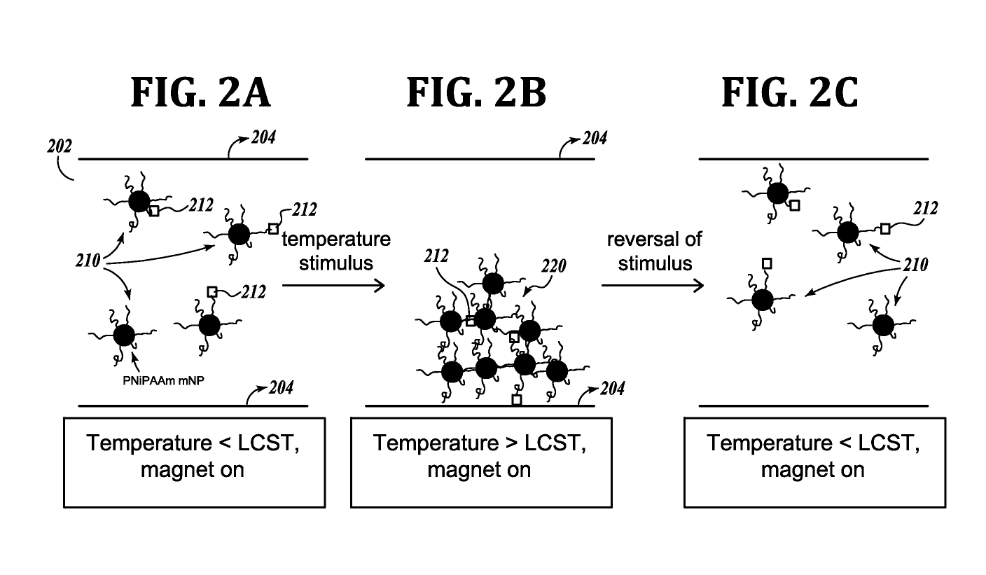

FIGS. 2A-2C are schematic illustrations of a representative method of capturing and releasing an embodiment of mNPs of the present disclosure. Referring to FIG. 2A, PNIPAAm mNPs 210 are contained in a vessel 202 having walls 204. The mNPs 210 are soluble and freely diffusive in the solution when the temperature is below the LCST of PNIPAAm mNPs. The size of PNIPAAm mNPs can provide the mNPs with low magnetophoretic mobility, so that they are not captured by an applied magnetic field below the LCST. The mNPs can thus diffuse and capture targets 212 as isolated nanoparticles below the LCST. Referring to FIG. 2B, after application of a temperature stimulus that is above the LCST of the mNPs, the mNPs aggregate to form aggregated mNPs 220. The aggregated mNPs are captured only when the temperature is raised above the LCST and when a magnetic field is applied (at the bottom vessel wall 204). Referring to FIG. 2C, when the temperature is lowered below the LCST, the reversible nature of the temperature-induced aggregation results in the release and redispersion of the captured aggregated mNPs and the diffusive re-entry of the mNPs 210 into the reaction solution.

Assays that Utilize Stimuli-Responsive Nanoparticles

The disclosure also provides assays for using the stimuli-responsive nanoparticle.

In one embodiment, the disclosure provides an assay for detecting a diagnostic target, including:

(a) contacting the diagnostic target with a plurality of stimuli-responsive mNPs, wherein each nanoparticle including a capture moiety having affinity toward the diagnostic target;

(b) forming nanoparticle conjugates by combining the diagnostic target with the stimuli-responsive mNPs;

(c) aggregating the nanoparticle conjugates by applying an external stimulus;

(d) further aggregating the nanoparticle conjugates by subjecting the aggregated nanoparticle conjugates to a magnetic field;

(e) regenerating the nanoparticle conjugates by removing the stimulus and the magnetic field; and

(f) analyzing the regenerated nanoparticles including the diagnostic target.

In the above method, forming nanoparticle conjugates by combining the diagnostic target with the stimuli-responsive mNPs provides a conjugate that includes a diagnostic target bound to the capture moiety. In the above method, regenerating the nanoparticle conjugates by removing the stimulus and the magnetic field provides released, free flowing nanoparticle conjugates in which the diagnostic target is bound to the capture moiety.

The regenerated nanoparticles including the diagnostic target can be analyzed with or without release of the diagnostic target from the nanoparticle.

The diagnostic target can be a molecule that is indicative of a diseased condition or an indicator of exposure to a toxin, or a therapeutic drug that has been administered to a subject and whose concentration is to be monitored. The diagnostic target can be any protein, antibody, or nucleic acid related to a disease. In one embodiment, the diagnostic target is an antibody against hepatitis B virus. In one embodiment, the diagnostic target is an antibody against hepatitis C virus. In one embodiment, the diagnostic target molecule is an antibody against AIDS virus. In one embodiment, the diagnostic target molecule is the malaria parasitic antigen, or the antiplasmodial antibodies, or the parasitic metabolic products, or the plasmodia nucleic acid fragments. In one embodiment, the diagnostic target molecule is an antibody against tuberculosis bacteria. In one embodiment, the diagnosis target molecule is a dengue fever virus or antibody. Methods, devices, and assays that use mNPs are described, for example, in U.S. Pat. Nos. 8,507,283 and 7,981,688, and PCT/US2011/035256, the disclosure of each of which is herein incorporated by reference in its entirety.

The following example is included for the purpose of illustrating, not limiting, the described embodiments.

### EXAMPLE

### Preparation and Characterization of Representative Stimuli-Responsive Magnetic Nanoparticles

In this example, the preparation, characterization, and use of representative temperature-responsive nanoparticles of the disclosure are described.

### Synthesis of Precursor Reagents

Iron oleate (Fe-oleate₃) was used as the iron source for stimuli-responsive magnetic nanoparticles (mNPs). The Fe-oleate₃ was prepared according to previously published methods. See, e.g., Park et al., Nature Materials, 3:891-895, 2004.

Briefly, sodium oleate (TCI, >97%) and iron (III) chloride hexahydrate (Sigma Aldrich, 97%) were dissolved in a mixed solvent system of hexanes, ethanol and deionized water. The solution was refluxed for 4 hours at 70° C., washed thrice in deionized water and dried to yield a viscous, dark red oil. Yield=75%. Composition was confirmed by ¹H-NMR on a Bruker Avance spectrometer operating at 300 MHz in CDCl₃.

In this example, the stimuli-responsive polymer responded to changes in temperature. The temperature-responsive, hydrophilic polymer is based on N-isopropylacrylamide (NIPAM) monomers and is synthesized with reversible addition fragmentation chain transfer (RAFT) techniques, as described, for example, in Chiefari, J. et al., Living Free-Radical Polymerization by Reversible Addition-Fragmentation Chain Transfer: The RAFT Process. Macromolecules, 31: 5559-5562, 1998. The molecular weight and molecular weight distribution were determined via size exclusion chromatography with multi- angle laser light scattering (Wyatt miniDAWN TREOS) and refractive index (Wyatt Optilab T-rEX) detectors. The polymer composition was confirmed by 1H-NMR, as described above.

Synthesis and Purification of mNPs

In a typical reaction, a round bottom flask was charged with 0.9 g of stimuli-responsive polymer and 50 mL of tetra(ethylene glycol) dimethyl ether (Sigma, ≧99%). The solution was heated at 100° C. for 30 minutes to dissolve the polymer. Then, 1.369 g of Fe-oleate₃ was added to the flask. The temperature was increased to 190° C. (heating rate ˜3.5° C./min) and held at 190° C. for 6 hours, during which the solution changed color from dark red to black. The reaction was cooled to <100° C. and precipitated from pentane. The precipitated mNPs were re-solubilized in tetrahydrofuran and precipitated from pentane for 2 additional times. After precipitation, the mNPs are dried in vacuo overnight. The dried mNPs are solubilized in deionized water to ˜4 mg/mL and then purified by tangential flow filtration (TFF) with a Vivaflow 50 100 kDa PES cartridge (Sartorius). After TFF, the mNPs are concentrated to >50 mg/mL and then lyophilized. The yield was 31±2.0% (n=6).

Characterization of mNPs

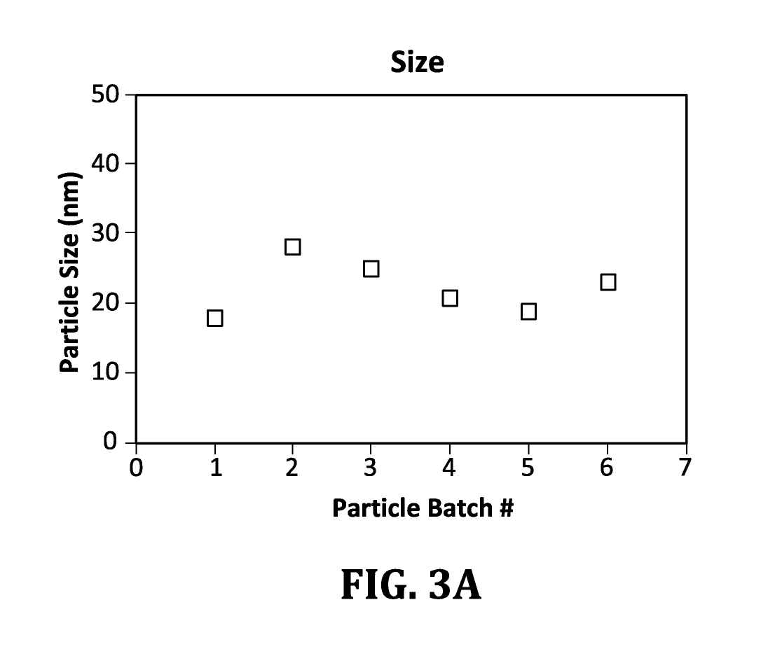

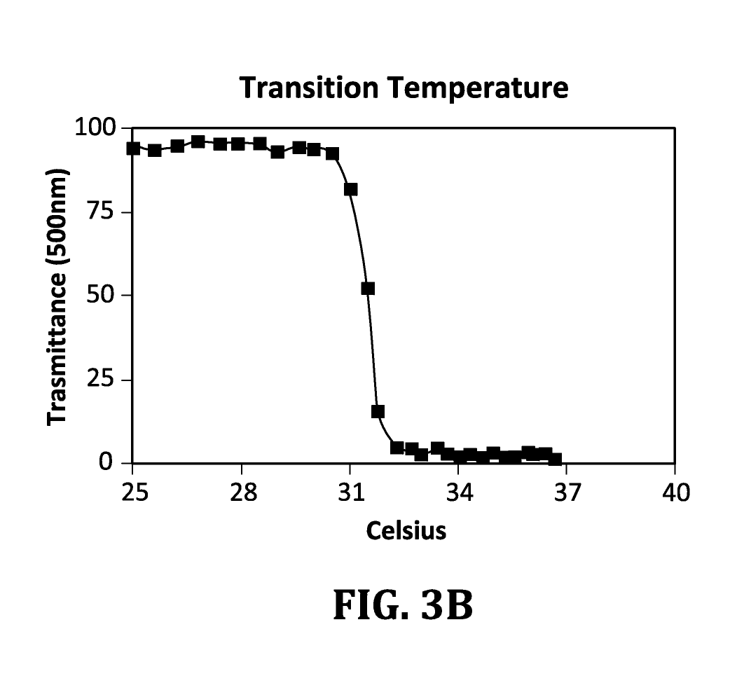

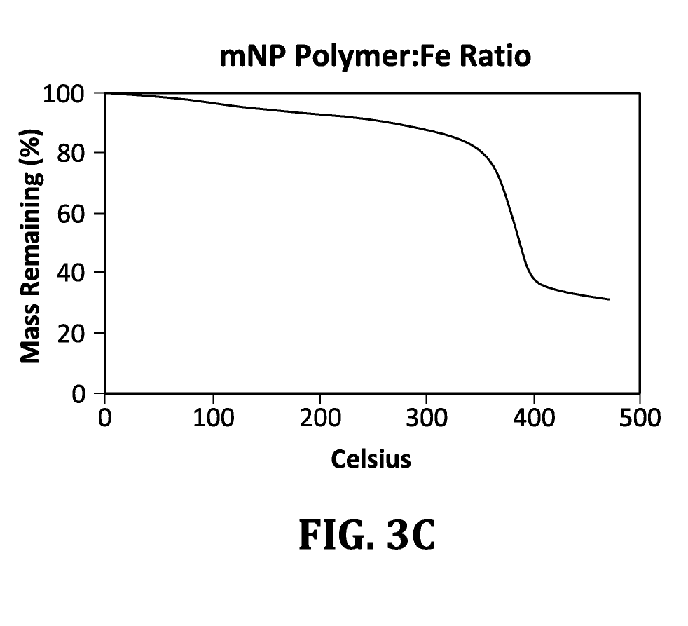

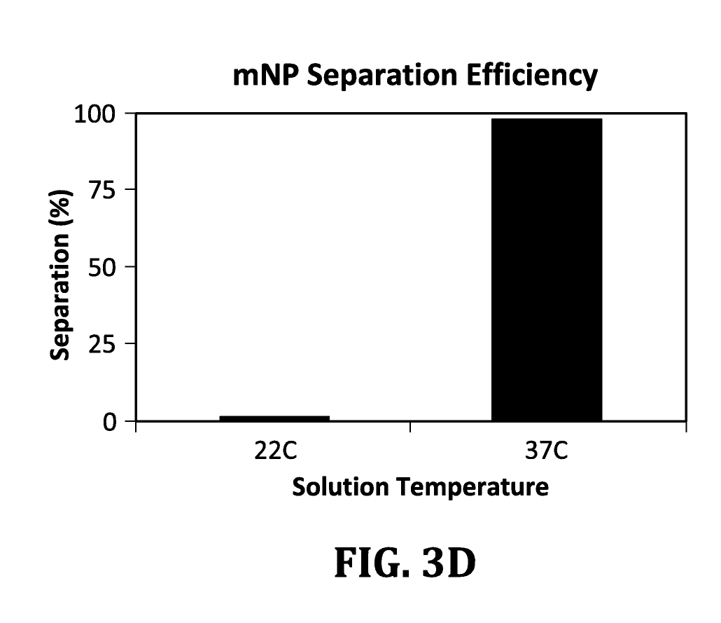

The hydrodynamic size of the mNPs was measured by dynamic light scattering (DLS) with a Brookhaven Instruments ZetaPALS instrument at 1 mg/mL in deionized water. The number-weighted average diameter for six different mNP batches was presented in FIG. 3A and Table 1.

The lower critical solution temperature (LCST) was measured by following the transmittance of a mNP solution with a UV-Visible spectrophotometer as the mNPs were heated from 25° C. to 37° C. (FIG. 3B). At the LCST (31° C.), the mNPs aggregated, resulting in a decrease in solution transmittance.

The polymer:iron mass ratio was measured by thermogravimetric analysis (TGA) on a TA Instruments TGA Q50 (FIG. 3C).

The mNP separation efficiency was a measure of how many mNPs could be separated from a solution after 2 minutes of exposure to a simple magnet. A mNP solution (2 mg/mL in 10 mM PBS with 5% serum) was exposed to 22° C. or 37° C. conditions for 2 minutes. The solution was kept at 22° C. or 37° C. and then placed in a custom-designed magnetic holder for 2 minutes. The absorbance (500 nm) of the supernatant was measured on a Tecan Safire₂ plate reader. The loss of absorbance was compared to a mNP control solution and quantified as the separation efficiency (FIG. 3D).

TABLE 1
Structural and functional properties of stimuli-responsive
mNPs (data presented as the mean ± standard deviation
for six different mNP batches, and small deviations indicate
reproducibility of mNP production process).

| Property | Measurement |
|---|---|
| Size | 22 nm ± 3.8 nm |
| 22° C. separation | 4.5% ± 3.3% |
| 37° C. separation | 98% ± 0.84% |
| LCST | 31° C. ± 0.85° C. |
| Polymer:Fe mass ratio | 2.0 ± 0.20% |

### Results

The Fe-oleate₃ complex composition was determined by ¹H-NMR, which showed appropriate shifts and integration values for oleic acid. The hydrophilic, stimuli-responsive polymer was 6.18±0.455 kDa with a polydispersity index of 1.03±0.007 (n=5), as measured by size exclusion chromatography. The low polydispersity index was a favorable characteristic of RAFT. Here, it indicated that the polymer was nearly monodisperse, which resulted in rapid and discrete transitions from hydrophilic polymers to hydrophobic aggregates in response to changes in temperature.

FIG. 3A shows the hydrodynamic diameter (number-weighted averages) of six different batches of mNPs, as measured by DLS. The diameters of these mNP batches ranged from 18 nm to 28 nm. The instrument error was approximately 5 nm. The LCST describes the temperature at which hydrophilic polymers aggregate into hydrophobic agglomerates, and is measured by the cloud point, or solution transmittance (FIG. 3B). The LCST of NIPAM-based polymers was typically around 32° C., which agreed with the mNP LCST values. Therefore, incorporation of the polymer with the mNPs did not affect the stimuli-responsive behavior of the polymer.

During TGA, dry samples are heated rapidly, which cause organic material to vaporize or combust, thus leading to a decrease in sample mass. Here, TGA was used to measure the amount of polymer incorporated around the inorganic iron oxide nanoparticle core of the mNPs. The mass loss at ˜100° C. was about 5% and represented the loss of water that was not removed by lyophilization. The polymer decomposition occurred over a broad range of temperatures (˜250-400° C.). The remaining mass was typically 32%, which represented the iron oxide core of the mNPs. The polymer:Fe mass ratio is shown in Table 1, and was calculated from the major decomposition mass loss and the remaining mass.

The separation efficiency (FIG. 3D) is an important functional property of the mNPs. Below the LCST at 22° C., the mNPs were soluble and too small for separation with a simple magnet (separation efficiency ˜5%). Above the LCST, the mNPs formed large aggregates that were easily separated with a simple magnet (separation efficiencies ˜98%). Therefore, the stimuli-responsive behavior of the mNPs translates to a useful functional characteristic.

FIGS. 3B-3D show data from a single representative mNP batch. The same properties (average±standard deviation) are shown in Table 1 from six different batches of mNPs. The small standard deviations show that the mNP production yields mNPs with reproducible structural and functional properties.

Stimuli-responsive mNPs (n=3) were also produced with NIPAM-based stimuli-responsive polymers that contained a proximal micelle-forming terminal group. However, these mNPs displayed poor structural and functional properties. One of the three batches was water-insoluble. The yield was much lower (18±4%). The mNP size was much larger (57±18 nm), which possibly explains the higher separation efficiency at 22° C. (8±4%). The lower LCST values (27±3° C.) are close to room temperature, which could allow the mNPs to aggregate during normal benchtop manipulations. Furthermore, the larger standard deviations indicated that these mNPs cannot be produced reproducibly, unlike those made with stimuli-responsive, hydrophilic polymers without micelle-forming terminal groups.

### CONCLUSION

Example 1 demonstrates the ease of synthesis and reproducibility of embodiments of stimuli-responsive mNPs of the present disclosure. The stimuli-responsive mNPs demonstrate superior characteristics when compared to stimuli-responsive mNPs made using stimuli-responsive polymers that contained a micelle-forming group at the proximal terminus.

While illustrative embodiments have been illustrated and described, it will be appreciated that various changes can be made therein without departing from the spirit and scope of the disclosure.

1. A process for making a stimuli-responsive magnetic nanoparticle, comprising:
providing a mixture comprising
a solvent having a boiling point of greater than 150° C. at atmospheric pressure,
a metal complex comprising a chelating agent coordinated to a metal cation of an element selected from Fe, Ni, Cr, Co, Gd, Dy, and Mn;
and a stimuli-responsive polymer; and
heating the mixture to provide a stimuli-responsive magnetic nanoparticle,
wherein the stimuli-responsive polymer does not comprise a terminal micelle-forming group.

2. The process of claim 1, wherein the solvent has a polarity index of greater than 2.4.

3. The process of claim 1, wherein the solvent is selected from the group consisting of diglyme, triglyme, tetraglyme, acetyl acetone, anisole, benzonitrile, cyclohexanone, N,N-dimethylaniline, N,N-dimethylformamide, dimethylsulfoxide, benzyl alcohol, cyclohexanol, diethylene glycol, n-heptanol, n-octanol, xylene, toluene, and any combination thereof.

4. The process of claim 1, wherein the solvent comprises oligoethylene glycol ethers.

5-6. (canceled)

7. The process of claim 1, wherein the chelating agent is selected from the group consisting of a C₈-C₂₈ fatty acid, a bipyridine, 4-vinyl pyridine, ethylene diamine, and ethylenediaminetetraacetic acid, and derivatives thereof.

8. The process of claim 1, wherein the chelating agent is oleic acid.

9. The process of claim 1, wherein the mixture comprises the metal complex at a concentration of from 5 mg/mL to 110 mg/mL.

10. The process of claim 1, wherein the stimuli-responsive polymer comprises polymers and copolymers of N-isopropylacrylamide substituted with a terminal functional group selected from the group consisting of a carboxylic acid, a primary amine, a secondary amine, a thiol, a hydroxyl, an aldehyde, a ketone, an azide, a hydrazide, and any combination thereof.

11. (canceled)

12. The process of claim 1, wherein the mixture comprises the stimuli-responsive polymer at a concentration of from 2 mg/mL to 75 mg/mL.

13. The process of claim 1, wherein the heating step is performed under an atmosphere comprising oxygen.

14. The process of claim 1, wherein the heating step comprises heating to a temperature of between 100° C. and 240° C.

15. (canceled)

16. The process of claim 1, wherein the heating step comprises refluxing the mixture for a duration of from 1 to 10 hours.

17. The process of claim 1, wherein the stimuli-responsive nanoparticle comprises a core comprising a magnetic metal oxide formed from the metal cation and wherein the stimuli-responsive polymer is coordinated to the core via a terminal functional group on the stimuli-responsive polymer.

18-19. (canceled)

20. The process of claim 1, wherein the stimuli-responsive nanoparticle responds to a stimulus selected from the group consisting of temperature, pH, light, electric field, and ionic strength.

21. The process of claim 1, wherein the stimuli-responsive nanoparticle has a hydrodynamic diameter of from 10 nm to 60 nm.

22. A stimuli-responsive magnetic nanoparticle, comprising:
a metal oxide core; and
a shell surrounding the metal oxide core, the shell comprising a stimuli-responsive polymer comprising a terminal carboxylate group,
wherein the terminal carboxylate group is directly coordinated to the metal oxide core and wherein the stimuli-responsive polymer does not comprise a terminal micelle-forming group.

23. The stimuli-responsive magnetic nanoparticle of claim 22, wherein the metal oxide core comprises a metal oxide selected from the group consisting of iron oxide, nickel oxide, nickel oxide, chromium oxide, gadolinium oxide, dysprosium oxide, and manganese oxide.

24. (canceled)

25. The stimuli-responsive magnetic nanoparticle of claim 22, wherein the stimuli-responsive polymer comprises polymers and copolymers of N-isopropylacrylamide.

26. The stimuli-responsive magnetic nanoparticle of claim 22, wherein the stimuli-responsive polymer comprises a terminus distal to the metal oxide core having a formula

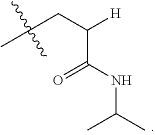 [SMILES: *CCC(=O)NC(C)C]

27. (canceled)

28. The stimuli-responsive magnetic nanoparticle of claim 22, wherein the nanoparticle comprises a stimuli-responsive polymer to metal oxide mass ratio of from about 1:1 to 3:1.
